(19) United States
(12) Patent Application Publication
(10) Pub. No.: US 2022/0037017 A1
(43) Pub. Date: Feb. 3, 2022

(54) REMOTE MEDICINE BASED ON VIDEO LINK AND SENSOR DATA

(71) Applicant: Healthcare Integrated Technologies Inc., Knoxville, TN (US)

(72) Inventors: Kenneth M. GREENWOOD, Davenport, FL (US); Scott Michael BORUFF, Knoxville, TN (US); Jurgen VOLLRATH, Sherwood, OR (US)

(21) Appl. No.: 17/391,928

(22) Filed: Aug. 2, 2021

Related U.S. Application Data
(60) Provisional application No. 63/103,408, filed on Aug. 3, 2020.

(51) Int. Cl. G16H 40/67; G16H 80/00; G16H 50/30; G16H 50/20; A61B 5/11; G08B 21/04

(52) U.S. Cl. CPC G16H 40/67; G16H 80/00; G08B 21/043; G16H 50/20; A61B 5/1117; G16H 50/30

(57) ABSTRACT

A method and system for performing telemedicine includes a real time video conference session between patient and physician coupled with patient physiology sensors for facilitating an initial diagnosis, followed by an ad hoc set of medical sensors to assist in defining a refined diagnosis.

### FIELD OF THE INVENTION

The invention relates to remote medicine. In particular it relates to a system and method for a physician to effectively diagnose a patient's ailments.

### BACKGROUND OF THE INVENTION

Telemedicine has become an important new approach to diagnosing and treating patients, which reduces the time and cost of a doctor visit and reduces exposure by patients and medical staff to pathogens.

### SUMMARY OF THE INVENTION

According to the invention there is provided a method for performing remote medicine (telemedicine), comprising receiving a request for a remote medical session, connecting the patient to a medical practitioner through a video link; establishing an initial diagnosis based on one or more of, a discussion between the medical practitioner and one or more of the patient, and an authorized support person (also referred to herein as a care-provider), and based on data from one or more physiological sensors that provide ongoing monitoring of the patient, and validating the initial diagnosis by capturing medical data using one or more medical sensors that the patient or a care-provider are instructed to apply or use with the patient for purposes of the session.

Following the session, the patient may be provided with confirmation of a session outcome and instructions for further action. The instructions may include one or more of: scheduling an in-person visit with a medical practitioner, and requesting one or more medical sensors to be delivered to the patient for future session monitoring.

The method may include providing the medical practitioner with copies of the discussion, data from any physiological sensors relating to the session, and data from the medical sensors; in the form of a dashboard, a video file, or a transcript of the discussion.

In a preferred embodiment, the discussion, and the physiological and medical sensor data are integrated into the electronic medical record of the patient.

Ongoing monitoring by physiological sensors may include continuous monitoring or capturing data at regular intervals or in response to identified events. The identified events can include events triggered by the patient, and anomalies detected by a physiological sensor.

The requesting of a medical sensor can be initiated in different ways, including a request by a medical practitioner placing an order for the medical sensors, or requiring the patient or care-provider to place an order for the medical sensors, or authorizing a third party who is present at the session to order or provide the medical sensors.

In one embodiment, the medical sensors are prepackaged for monitoring a specific condition, such as sleep apnea, or cardio vascular problems, or may be packaged to include the sensors most suitable to address the issues that a particular patient is dealing with.

An artificial intelligence system may be used for identifying anomalies in physiological sensor data. Data from the physiological sensors and data from the medical sensors may be processed by the artificial intelligence system to identify correlations between at least one of: time-related physiological sensor data and medical sensor data, and medical sensor data with previously identified events, in order to improve the diagnosis of a condition and to provide refined training data for the artificial intelligence system.

The process of identifying correlations may include identifying anomalies in the data of one or more sensors during an identified event or at a time interval preceding or following an identified event.

In accordance with the invention, the request for a remote medical session may be initiated by a medical practitioner, by the patient, by a third party or artificial analysis (AI) system in response to the data correlation between sensors, which is indicative of an identified event, or at defined intervals as part of a preventative health check-up.

For safety and privacy purposes, the identities of the patient and medical practitioner are preferably authenticated prior to connecting the patient and medical practitioner.

Further, according to the invention, there is provided a system for providing telemedicine, comprising: a physician communication platform, a patient communication platform, wherein the communication platforms are adapted to conduct a video communication session between the patient and physician communication platforms, one or more continuous patient monitoring devices, one or more medical sensors, and a server system for collecting data from the communication session between physician and patient, data from the one or more patient monitoring devices (also referred to herein as physiological sensors), and data from the one or more medical sensors, wherein the server system includes a processor connected to memory which includes an algorithm for analyzing the data for events associated with one or more physiological conditions (also referred to herein as flagging events).

The algorithm may form part of an artificial intelligence network, and may be configured to identify anomalies and events (e.g. a patient falling) identified by any of the patient monitoring devices over time, and to correlate such anomalies with data for the same time frame or a related prior or subsequent timeframe from the same or other monitoring devices.

The AI system may include data inputs from all of the medical sensors to identify events and anomalies during a session, and correlations with the data in any of the other sensors and monitoring devices, thereby defining a flagging event.

The algorithm may be arranged to trigger a communication session if a flagging event is identified. A flagging event may include pre-defined events such as a falling event, or an anomaly in a monitoring device (physiological sensor) that exceeds a predefined threshold or that correlates with anomaly data from at least one other monitoring device for the same or a related time-frame, i.e., is corroborated by at least one other monitoring device.

The physician communication platform may include a screen, a processor, and access to the internet with a browser for accessing a portal that allows the physician to conduct a video call with the patient and access data from any of the patient monitoring devices and medical sensors, and download the data from the video call and the monitoring devices and sensors. In order to authenticate him or herself, the physician and the patient may be required to sign in or register via a software app or website, and provide the necessary authenticating information.

The physician and user communication platforms may comprise pre-existing hardware, e.g., smart phone, tablet, laptop, desktop computer, etc. Each communication platform is configured to the unique characteristics and attributes of the physician (e.g. name, address, type of medical provider, specialization, plus supporting credentials, etc.) or of the patient, respectively (e.g., name, address, date of birth, etc.) by providing a sign-on or registration session with a capture page for capturing the physician's or user's details. Separate registration sessions may be provided for physicians and patients.

### BRIEF DESCRIPTION OF THE DRAWINGS

FIG. 1 is a depiction of one embodiment of the implementation of a system of the invention;

FIG. 2 is a flow chart defining the logic of one embodiment of an anomaly detection algorithm implemented in an AI system;

FIG. 3 is a flow chart defining the logic of one embodiment of an anomaly detection and corroboration algorithm implemented in an AI system, and

FIG. 4 shows one embodiment of a physician portal.

### DETAILED DESCRIPTION OF THE INVENTION

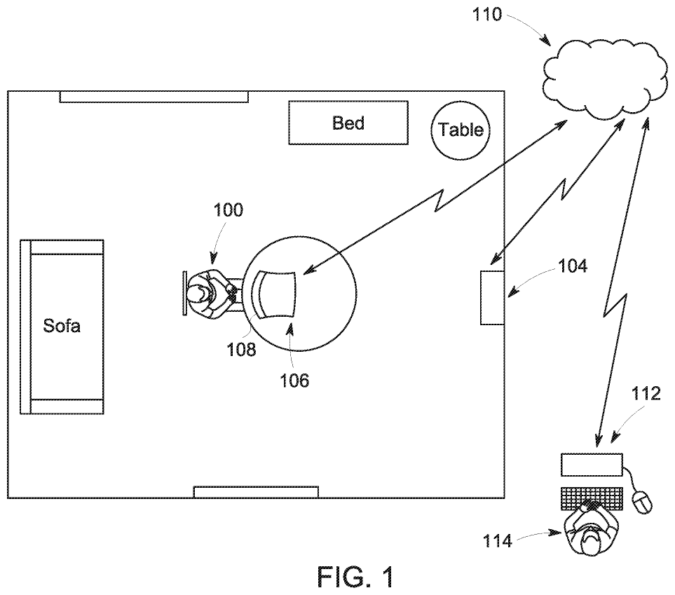

One embodiment of a system for implementing the present invention is shown in FIG. 1.

The patient 100 will in most circumstances be residing at home or at some form of care facility such as a continuing care retirement community (CCRC), which will for purposes of this application and for ease of reference be referred to as a suite 102. In the present embodiment the suite includes a physiological sensor (also referred to as a patient monitoring system) in the form of a camera 104 for monitoring the patient, as well as communications system 106 with a voice user interface, with local or remote processing such as a voicebot, similar to Alexa by Amazon or ElliQ by Intuition Robotics, to allow the patient to make a hands-free request for help or to communicate with friends and family.

It will be appreciated that in other embodiments the physiological sensors may include one or more of a variety of devices for either continuously or regularly monitoring the activities or movements or other physiological parameters of the patient, e.g. some form of wearable device (e.g. Life Alert pendant, or Fitbit watch), or other types of ambient sensors such as radar, lidar, microphones, etc., that may be mounted on a wall of the suite 102.

The communications system 106 includes a display screen 108 to provide a visual interface, which may comprise a pre-existing screen, e.g., smart phone, tablet, laptop, etc., or an ad hoc user interface screen forming part of the communications system 106.

The camera 104 may be implemented to continuously monitor the patient and communicate video data to a central server or cloud server system 110, where at least part of the data is captured in memory for further processing, evidence retention and artificial intelligence (AI) system learning, as is discussed in greater detail below. In some embodiments, the camera may include a processor to perform rudimentary analysis of the image data, for purposes of identifying a predefined event, such as a fall, in which case it may limit the communication of data to a server, to said event data. The camera may also include a buffer for storing a certain amount of video data (frames) preceding an event to allow, for example, the last 30 seconds of video data prior to an event to be captured and transmitted to the server system.

In response to the detection of an event or based on a patient request, or based on a scheduled medical check-up session, a communication link (communication session) may be established between the patient's communication system 106 and a remote communication system 112, which is accessible by a medical practitioner 114, e.g., by the patient's regular care physician. The medical practitioner 114 (who for ease of reference will be referred to herein simply as the physician) can then discuss the situation with the patient 100 and make an initial diagnosis based on a discussion of the patient's symptoms and events leading up to the communication session.

The remote communication system 112, in this embodiment comprises a desktop computer that is connected to the Internet and accesses a website that is configured as a portal that the physician 114 initially registers on (providing his or her profile details to create a physician-specific user ID that is password protected. Subsequently during a communication session the physician logs onto the portal by verifying his or her user ID or password. In this embodiment, the physician's identity is password verified but could include other forms of authentication, e.g. using a voice print or facial recognition.

The patient in this embodiment similarly authenticates him or herself during an initial log-on/registration session to the server 110, which includes providing personal identifying details that will form part of a password protected user ID for the patient.

In this embodiment the physician is presented with a different portal to that of the patient. The physician portal allows the physician 114 to conduct the video consultation with the patient, as well as review any physiological sensor data that is available (in this case data from the camera 104).

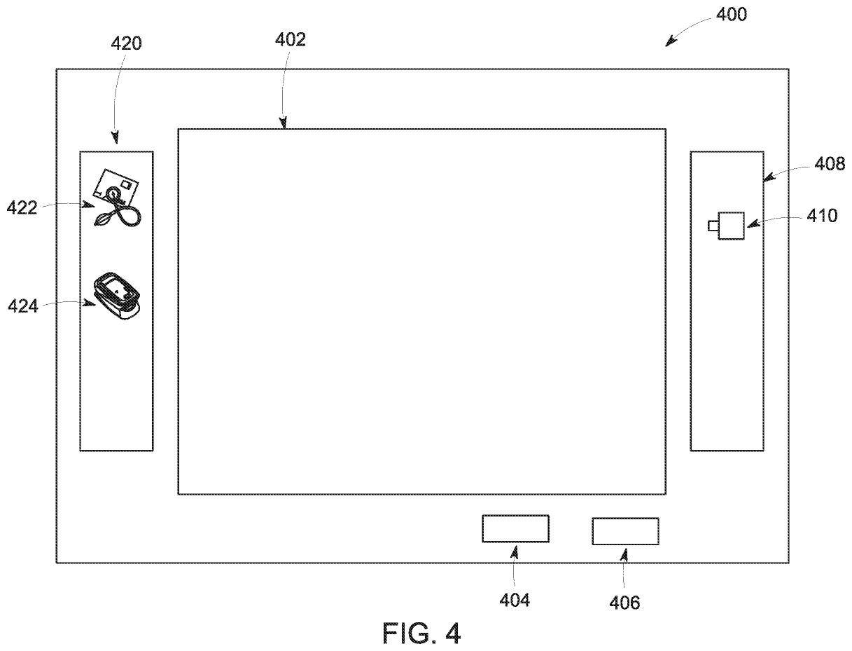

FIG. 4 shows one embodiment of a physician portal accessed by the physician during a session with a patient.

The portal 400 includes a video screen region 402 that can be collapsed and expanded during a session. A connect button 404 and disconnect button 406 are included in this embodiment to initiate a session and terminate a session, respectively. The right-hand margin defines a physiological sensor region 408 for displaying icons of any physiological sensor associated with the patient 108.

The sensor icons populate the region 408 only insofar as there are physiological sensors connected to the server 110. In this embodiment there is only one physiological sensor, in the form of the camera 104, which is depicted as a camera icon 410.

The left-hand margin defines a medical sensor region 420 for displaying icons of medical sensors attached to or used on a patient.

Thus, in this embodiment, the physician 114 can request the patient or a care provider (e.g., a relative or nurse at a care facility) to attach or apply one or more medical sensors to the patient in order to provide additional information to the physician for making a better diagnosis. For instance, if the patient is complaining of chest pain, the physician 114 may wish to monitor heartbeat, blood oxygen levels, and blood pressure, and can request specific medical sensors, e.g., a blood pressure cuff and blood oximeter reader, to be attached to the patient 100 in order to capture additional information. Once the medical sensors are configured and communicate with the server 110, a blood pressure cuff icon 422 and a blood oximeter reader icon 424 appear on the physician portal in the medical sensor region 420.

In a preferred embodiment the patient's communication system 106 is configured to communicate with the physiological sensors 104 and medical sensors 422, 424, e.g., through USB ports or Bluetooth connection in order to capture the medical data from the physiological sensors and medical sensors and relay the data to the server 110 and make it available to the physician via the portal on the remote communication system 112.

By clicking on the icons 410, 422, 424 the physician can change screens or open a sub-screen in the video screen 400 region, in order to either view the video data captured by the camera 104, or review data captured by the medical sensors 422, 424.

In the above embodiment the medical sensors were chosen to diagnose a potential heart issue, and comprised a set of electrodes for capturing blood pressure and blood oxygenation levels. However, it will be appreciated that the physician could request data from additional sensors, e.g., electrocardiogram (ECG) sensors. In another case the physician may request other data sensors to be used or applied to the patient in order to investigate or diagnose other medical conditions.

The discussions between the physician and patient may be made available to the physician as a sound file and a video file. The sound file may be transcribed by transcription software at the server 110 and made available on the portal 400 for the physician to download and add to the patient's electronic medical records (EMR).

In one embodiment the various data files and transcript are reconfigured at the server 110 to conform with the format required by the physician's EMR system or to comply with a set of standards for integration into any one of a variety of EMR systems.

Thus, the discussions between the physician 114 and patient 100 may in one embodiment be transcribed and integrated via an application program interface (API) into the EMR together with the video files, sound files, or other data files from the physiological sensor 104 and medical sensors (depicted in Figure two by icons 222, 224).

As mentioned above, in one implementation of the server system will capture in memory at least part of the sensor data and communication between the physician and the patient.

This serves not only to assist the physician and supplement the patient's EMR record, it provides improved insight into the patient by capturing the data, further processing the data for analysis, and for refining an artificial intelligence (AI) system (as is discussed in greater detail below). It also allows retention of records for future evidence, e.g., in legal proceedings.

Thus, in this embodiment, the server 110 includes a processor and memory that includes an algorithm defining an AI system for the sensor data to identify anomalies in the data compared to previous data, and for time stamping data that corresponds to an anomaly. If the anomaly exceeds a predefined threshold (e.g. a severe stumble by the patient) or is of a certain type (such as the detection of a fall) the anomaly is registered as a flagging event (also referred to herein as an emergency event). A flagging event may also be registered if an anomaly is corroborated by at least one other sensor for the same or a related time-frame, e.g., 15 minutes before or after an anomaly being detected by the first sensor. The logic for comparing data from other sensors for the same or adjacent time-frames to an anomaly may serve not only to corroborate a first sensor but may also serve to explain the anomaly.

In one embodiment, involving two physiological sensors: a video camera, e.g., the camera 114 and a microphone may both be monitoring the patient. Video data received from a camera, e.g., the camera 104, may be parsed into frames, and audio data captured from the microphone is similarly parsed into digital signal amplitudes and signal frequencies that are each separated by a time interval Δt wherein three successive time intervals from each of the sensors (in this case camera, microphone amplitude, microphone frequency) are fed into an artificial neural network to identify changes for each sensor feed over time and related to corresponding changes for the same or a related time frame e.g., for time t from the camera related to time t+x·Δt for the microphone (amplitude and frequency), where x can for instance be 1, 2, 0, −1, −2.

As indicated above, the present invention involves identification and analysis of anomalies. In one embodiment, the anomaly analysis is implemented in software and involves logic in the form of machine readable code defining an algorithm or implemented in an artificial intelligence (AI) system, which is stored on a local or remote memory (as discussed above), and which defines the logic used by a processor to perform the analysis and make assessments.

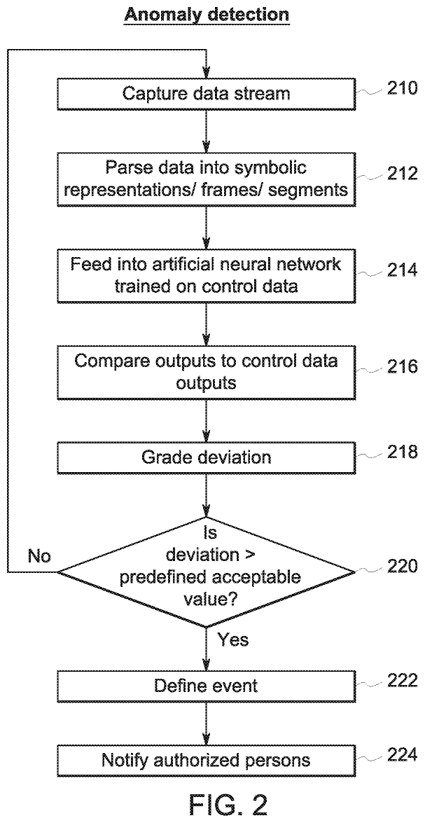

One such embodiment of the logic based on grading the level of the anomaly to determine if it surpasses a predefined threshold, is shown in FIG. 2, which defines the analysis based on sensor data that is evaluated by an Artificial Intelligence (AI) system, in this case an artificial neural network. Data from a sensor is captured (step 210) and is parsed into segments (also referred to as symbolic representations or frames) (step 212). The symbolic representations are fed into an artificial neural network (step 214), which has been trained based on control data (e.g. similar previous events involving the same party or parties or similar third-party events). The outputs from the AI are compared to outputs from the control data (step 216) and the degree of deviation is graded in step 218 by assigning a grading number to the degree of deviation. In step 220 a determination is made whether the deviation exceeds a predefined threshold, in which case the anomaly is registered as an event (step 222) and one or more authorized persons is notified (step 224) if the event qualifies as an emergency event based on the grading number.

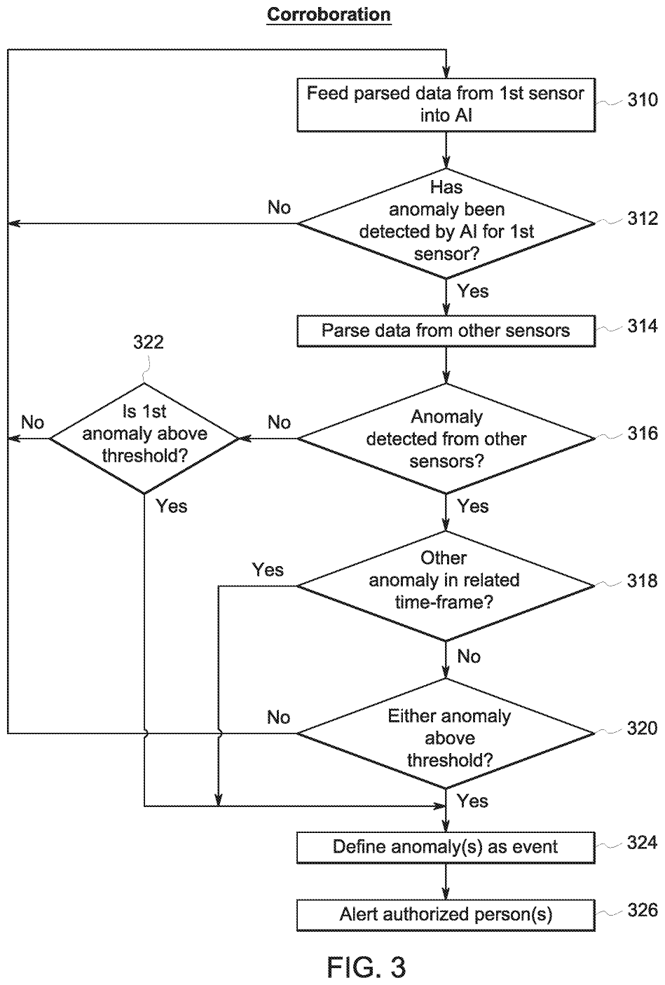

Another embodiment of the logic in making a determination, in this case, based on grading of an anomaly and/or corroboration between sensors is shown in FIG. 3.

Parsed data from a first sensor is fed into an AI system (step 310). Insofar as an anomaly is detected in the data (step 312), this is corroborated against data from at least one other sensor by parsing data from the other sensors that are involved in the particular implementation (step 314). In step 316 a decision is made whether any of the other sensor data shows up an anomaly, in which case it is compared on a time scale whether the second anomaly is in a related time frame (which could be the same time as the first sensor anomaly or be causally linked to activities flowing from the first sensor anomaly) (step 318). If the second sensor anomaly is above a first threshold deviation (step 320) and thus corroborates the first sensor, or similarly, even if there is no other corroborating sensor data, if the anomaly from the first or any other sensor data exceeds a second threshold deviation (step 322), the anomaly captured from either of such devices triggers an emergency event (step 324), which alerts one or more authorized persons (step 326).

According to one aspect of the invention, as more sensors (physiological sensors and medical sensors) are added the data is fed into neural networks with larger numbers of inputs, and as an event is confirmed as a positive diagnosis by a physician the corresponding data is fed back as part of an ongoing learning phase for the neural network.

This allows future events associated with a likely positive diagnosis to become the triggers for initiating a session between the patient and the physician.

It will be appreciated that references to the patient initiating a session with a physician includes an initiation by a person acting on the patient's behalf, such as staff at a CCRC or a relative of the patient.

Thus, the present system allows an initial diagnosis to be made based on the video interaction between the patient and the physician, coupled with the physiological patient sensors, which monitor autonomously and, in many cases, continuously, e.g. the camera 114 (FIG. 1). In order to refine the initial diagnosis, the present invention allows the physician to drill down to confirm or refute possible medical conditions by having the patient attach or apply specific medical sensors. In one embodiment, these may be provided by staff at a CCRC or may have been issued to the patient following a past emergency, e.g., at time of discharge from a hospital.

In one embodiment the system may identify the occurrence of a flagging event and may automatically contact various entities such as the physician, staff members at a facility, medical device suppliers, and/or family members, depending on the nature of the event.

The present invention also contemplates that in one embodiment a medical device supplier will be summoned to the suite of the patient and may assist in the diagnosis session by hooking up designated medical sensors, and/or may teach the patient how to connect or use the sensors for the current or future sessions. By having the medical device supplier present with a broad range of sensors or sensor kits it ensures that the necessary medical sensors are available during the session, allowing the physician to confirm an initial diagnosis, and allowing the physician to issue a script for the necessary sensors for future monitoring of the patient, which can then be executed by the medical device supplier in real time. This also ensures that the patient is taught how to use the sensors for future sessions. In one embodiment the sensors may be pre-packaged to deal with specific ailments or conditions, or may be packaged ad hoc, following a session based on the needs perceived by the physician for the particular patient.

The details of the sensors included in the package provided to a patient may be visually depicted on the box of the package for easy visual confirmation by the physician during a session, and may be automatically added to the physician's EMR by ensuring plug-and-play capabilities between the sensors and the patient communication system.

While the present invention has been described with reference to particular embodiments with specific physiological and medical sensors, it will be appreciated that different sensors and different configurations of the communication systems and server system can be implemented without departing from the scope of the invention.

What is claimed is:

1. A method for performing remote medicine (telemedicine), comprising:
receiving a request for a remote medical session,
connecting the patient to a medical practitioner through a video link,
establishing an initial remote diagnosis based on one or more of:
a) a discussion, via the video link, between the medical practitioner and one or more of: the patient, and an authorized support person (also referred to as a care-provider), and
b) data from one or more physiological sensors that provide ongoing monitoring of the patient, and
validating the initial remote diagnosis by capturing medical data using one or more medical sensors that the patient, a care-provider, or other person is instructed to apply or use with the patient as part of the remote medical session.

2. The method of claim 1, wherein, following the validation of the initial remote diagnosis, at least one of the following follow-up procedures are invoked: the patient is required to attend an in-person visit with a medical practitioner, and one or more medical sensors are delivered to the patient for future remote medical sessions.

3. The method of claim 1, wherein the medical practitioner is provided with copies of the discussion, data from any physiological sensors, and data from the medical sensors for inclusion in an electronic medical record of the patient.

4. The method of claim 1, wherein ongoing monitoring by physiological sensors includes continuous monitoring, or capturing data at regular intervals, or in response to identified events.

5. The method of claim 4, wherein the identified events include events triggered by the patient, and anomalies detected by a physiological sensor.

6. The method of claim 5, wherein an artificial intelligence system is used for identifying anomalies in physiological sensor data.

7. The method of claim 6, wherein data from the physiological sensors and data from the medical sensors is processed by the artificial intelligence system to identify correlations between at least one of: physiological sensor data and medical sensor data for a related time frame, and medical sensor data with previously identified events, in order to improve the diagnosis of a condition and to provide refined training data for the artificial intelligence system.

8. The method of claim 7, wherein the process of identifying correlations includes identifying anomalies in the data of one or more sensors during an identified event or at a time interval preceding or following an identified event.

9. The method of claim 6, wherein the request for a remote medical session is initiated by a medical practitioner, by the patient, by a third party, by the artificial intelligence system in response to an anomaly, or at defined intervals as part of a preventative health check-up.

10. A system for providing telemedicine, comprising:
a physician communication platform,
a patient communication platform, wherein the communication platforms are adapted to conduct a video communication session between the patient and physician communication platforms,
one or more physiological sensors (also referred to as patient monitoring devices) that perform ongoing monitoring of the patient,
one or more medical sensors, and
a server system for collecting data from the communication session between physician and patient, data from the one or more physiological sensors, and data from the one or more medical sensors, wherein the server system includes a processor connected to memory which includes machine-readable code defining an algorithm for analyzing the data from at least one of: the data from a physiological sensor, and data from a medical sensor, to identify a physiological conditions (also referred to herein as a flagging event).

11. The system of claim 10, wherein the algorithm forms part of an artificial intelligence (AI) network, which is configured to identify anomalies and events in the data of the physiological sensors over time.

12. The system of claim 11, wherein anomalies detected by the AI network are correlated with data for the same time frame or a related prior or subsequent timeframe from the same or other physiological sensors.

13. The system of claim 11, wherein the AI network includes data inputs from the medical sensors to identify anomalies in the medical sensor data during a session, and correlations with anomalies in the physiological sensors, thereby defining a flagging event.

14. The system of claim 13, wherein a flagging event includes a pre-defined event such as a falling event, or an anomaly in the data from a physiological sensor that exceeds a predefined threshold, or an anomaly in the data from a physiological sensor that correlates with an anomaly in the data from at least one other physiological sensor for the same or a related time-frame.

15. The system of claim 12, wherein the algorithm includes logic to trigger a communication session if a flagging event is identified.

16. The system of claim 10, wherein the physician communication platform includes a screen, a processor, and access to the internet, and a portal accessible through a browser or an app that allows the physician to conduct a video call with the patient and access data from any of the physiological sensors and medical sensors, and download the data from the video call and sensors.
